(19) United States
(12) Patent Application Publication
(10) Pub. No.: US 2024/0173040 A1
(43) Pub. Date: May 30, 2024

(54) SURGICAL DRILLING INSTRUMENT

(71) Applicant: MIMEO MEDICAL GMBH, Filderstadt (DE)

(72) Inventor: Frank HEUER, Filderstadt (DE)

(21) Appl. No.: 18/552,374

(22) PCT Filed: Mar. 25, 2022
(86) PCT No.: PCT/EP2022/058031

§ 371 Date: Sep. 25, 2023

(30) Foreign Application Priority Data
Mar. 26, 2021 (DE) 10 2021 001 608.4

(51) Int. Cl. A61B 17/16

(52) U.S. Cl. CPC A61B 17/1631; A61B 17/1628

(57) ABSTRACT

A surgical instrument for drilling into bone or bone fragments is disclosed having a drill section, a gear section and a longitudinal shaft section located therebetween, having a first and a second shaft element. The second shaft element is supported at least partially in the first shaft element, and each shaft element performs a cyclic translational movement along the longitudinal axis for drilling into the bone.

### STATE OF THE ART

Various drilling instruments for opening bones are known from the prior art, which are used to this end to prepare the bone for the use of implants, such as for example osteosynthesis screws. However, all drilling instruments work according to a rotational principle, in which a drill bit is rotated about an axis. This generates exclusively circular openings in the bone.

The prior art discloses implants (e.g. DE102019000965A1, WO2004107957A2, WO03032853A2) which preferably have a non-circular design so that they benefit from their biomechanical advantages on account of their shaping. However, there is currently no drilling instrument to prepare the implantation channel for such implants. So far, blade-like implants have been driven by striking into the bone, which can lead to clinical complications. A drilling instrument suitable for this purpose would have to be able to create a non-circular drilling channel and, in the case of blade-like implants, the drilling channel would even have to have a helical course along the drilling depth.

### DESCRIPTION OF THE INVENTION

The task is achieved through the drilling instrument (1) according to the invention presented herein.

For the surgical drilling instrument (1) according to the invention, coordinate references referring to the space are defined, such as for example a proximal direction (101), a distal direction (102), which develop along a central axis (103). Starting from the central axis (103) to the outside, both a radial expansion (104) and a circumferential direction (105) are defined.

The surgical instrument (1) according to the invention is suitable for drilling into bones or bone fragments and consists of a drill section (5), a gear section (2) and a longitudinal shaft section (3) located therebetween having a first (30) and a second shaft element (40). As a result, the second shaft element (40) is supported at least partially in the first shaft element (30). For drilling, each shaft element performs a cyclic translational movement (130, 140) along the longitudinal axis (103).

Preferably, the first shaft element (30) has a concentric opening (34) at least partially along the longitudinal direction (103), and the second shaft element (40) has a concentric outer wall (43) at least partially along the longitudinal direction (103). As a result, it is advantageous if the concentric outer wall (43) of the second shaft element (40) is inserted into the concentric opening (34) of the first shaft element (30). Thus, both shaft elements are oriented and supported along the central axis (103) relative to one another without additional means.

For such a mounting, it is advantageous for the first shaft element (30) to be designed at least partially tubular, and for the second shaft element (40) to be designed at least partially tubular or as a full-round rod. A tubular or full-round shape has the advantage that the shaft elements (30, 40) can be produced with the aid of a lathe, which brings about time-saving effects and thus also cost savings.

The gear section (2) consists substantially of a rotor (10) with a central opening (13) and of the two shaft elements (30, 40) supported therein. As a result, both shaft elements (30, 40) each form a cam disc (310, 410) at their proximal end (101). It is characteristic of the configuration according to the invention that the first and second shaft elements (30, 40) each form a cam disc (310, 410), and the cam disc of the second shaft element (410) is arranged in the proximal direction (101) towards the cam disc of the first shaft element (310). This is therefore a double-stage cam disc gear. Such an arrangement has the advantage that the parts are easier to produce and can also be disassembled at any time.

To convert a rotational movement (110) of the rotor (10) into a translational movement (130, 140) of the shaft elements (30, 40), the rotor (10) must have at least two pins (1031, 1041 and/or 1032, 1042), wherein at least one pin (1031 and/or 1032) is in engagement in the recess of the first cam disc (313, 312) and at least one further pin (1041 and/or 1042) is in engagement in the recess of the second cam disc (413, 412). A twist of the rotor (110) with the pins (1031, 1032, 1041, 1042) causes the pins to deflect (130, 140) the shaft elements (30, 40) due to the sinusoidal curved lines (312, 412). They are displaced along the longitudinal axis (103).

If only one pin per cam disc is engaged, a tilting moment occurs on the shaft element. This can lead to a one-sided loading, increased wear and thus jamming. In order to prevent this, it is advantageous if this tilting moment is prevented through another opposite pin. More specifically, in an advantageous embodiment, the rotor (10) has at least two circumferentially opposite pins (1031, 1032) that are in engagement in the recess of the first cam disc (312), and the rotor (10) has at least two further circumferentially opposite pins (1041, 1042) that are in engagement in the recess of the second cam disc (412).

Only the mechanical solution of inserting the shaft elements into each other allows two cam discs to be placed consecutively with continuous curved lines. This allows to the design to meet several mechanical requirements, such as: reduction of a jamming through opposite pins, dismountability, good cleanability, simplified production and reduced wear.

In an alternative embodiment, a counter-rotating cyclic translational movement of both shaft elements (130, 140) can also be generated in such a way that the sine waves (312, 412) of both cam discs (310, 410) are provided in phase synchronism. A counter-rotating motion may then be induced by placing the pins 1031 and 1041 as well as the pins 1032 and 1042 at an angle to one another in the circumferential direction. This angle and the number of the sinusoidal periods in the circumferential direction then determine the phase shift or the measure of the counter-rotation of the translational movement (130, 140, 150).

The surgical instrument (1) can be driven via the rotor (10), which has a proximally extending shaft area (12), which provides a coupling connection (11) which is suitable to this end for transmitting a torque to the rotor (110). Handgrips or rotating drives, e.g. electrical, electromechanical, mechanical, pneumatic, hydraulic or other types of drives, can be releasably connected to the surgical instrument (1) at this coupling connection (11). It is important that these drives or handgrips are suitable for use in surgery and meet the requirements for cleanability and sterilizability.

To adjust an orientation (108) of the drill section (5) with respect to the bone, it is necessary for a stator (20) to be provided. With the orientation of the stator (106), the orientation of the drill bit (108, 50) can be adjusted, varied, and/or kept constant. For this purpose, it may be helpful if an indicator (25) is provided on the stator (20) and which indicates the orientation of the drill bit (51, 106, 108). As a result, it is irrelevant whether the indicator (25) is a marking, notch, inscription, relief, a material application/or removal or an additional pointer element is provided.

For manually holding and adjusting the drill bit orientation (108) with the aid of the stator (106), it is important that at least one handle (24) is provided on the stator (20). The handle is also to be understood as referring to structures which are suitable to this end for holding the stator (20). These also include, for example, notches, grooves, thickenings, increased roughness, recesses, individually protruding handle elements or also openings (28). The stator (20) may also be part of a housing not shown herein.

In the preferred embodiment, the shaft elements (30, 40) are provided at least partially as a full-round or tube. For the shaft elements (30, 40) not to twist against each other, it is advantageous for the shaft elements (30, 40) to be in contact to one another directly (39, 49) or indirectly (37, 47, 21, 22) along the longitudinal axis (103) on at least one section so that a rotation of the shaft elements (30, 40) against each other is thus excluded. Furthermore, it is also important that no twist of the shaft elements (30, 40) with respect to the stator (20) can take place. For this purpose, a surface or projection (37, 47), which is in contact with the stator (20), must be provided on at least one of the shaft elements (30, 40), such that the shaft element (30 and/or 40) with the stator (20) are always at the same angle (106, 107) to one another in the circumferential direction.

So that the surgical drilling instrument (1) according to the invention can be guided by means of a guide wire (60) and can also be used for minimally invasive procedures, it is advantageous if the entire drilling instrument (1), including the drill section (5), is continuously cannulated (48, 18, 536, 546).

Drilling is carried out with a translationally oscillating drill section (5), wherein the drill section (5) consists of at least two separate drilling elements (53, 54) and these drilling elements are connected to the shaft elements (30, 40). As a result, each drilling element (53, 54) is deflected individually by the drilling instrument (1). The drilling elements have rasp teeth and, in combination, preferably a drill bit (50) tapering to the distal bit. Drilling is carried out with a cyclic translational rasping movement of the drilling elements (53, 54), simultaneously drilling and clearing material from the channel.

The advantage of the instrument (1) according to the invention is that the drilling elements (53, 54) in combination generate openings in the bone that have a non-round cross section. Furthermore, it is also conceivable for the opening in the bone to deviate from the central axis (103) along the depth and, for example, to follow a curvature. For blade-like implants, it is necessary and also possible for the drilling elements (53, 54) to form in combination a blade shape (52). When drilling with a translationally oscillating blade (52), it is then also possible to generate openings in the bone, wherein the opening has a first cross section and a second cross section at two different depths, and the orientations of both cross sections are different from one another.

### BRIEF DESCRIPTION OF THE DRAWINGS

FIG. 1 shows an oblique view of the drilling instrument according to the invention,

FIG. 2 shows an oblique view of an exploded view without a drill section,

FIG. 3 shows a side view of the drilling instrument according to the invention, as well as a detailed representation in section,

FIG. 4 shows a side view of the shaft elements with representation of the development of the cam discs,

FIG. 5 shows an alternative embodiment with three or more shaft elements,

FIG. 6 shows an oblique view of the drill instrument according to the invention with the demonstration that the drill section can be releasably connected,

FIG. 7a, b show detail and exploded view of the drill section

FIG. 8a-d show various views of the drill section,

FIG. 9a, b show alternative embodiment of the drill section with cannulation and guide wire,

FIG. 10 shows an alternative embodiment of the drilling instrument,

FIG. 11 illustrates an embodiment of FIG. 10 without a stator for better visibility,

FIG. 12a-c shows release mechanism for easy disassembly of the gear section for processing the instrument,

FIGS. 13a, b show an individual view of the alternative embodiment of FIG. 10.

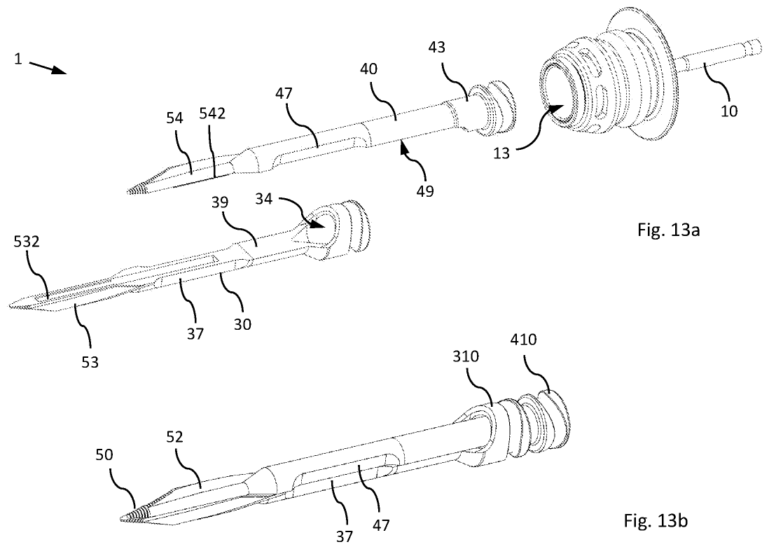

### DESCRIPTION OF THE PREFERRED EMBODIMENTS

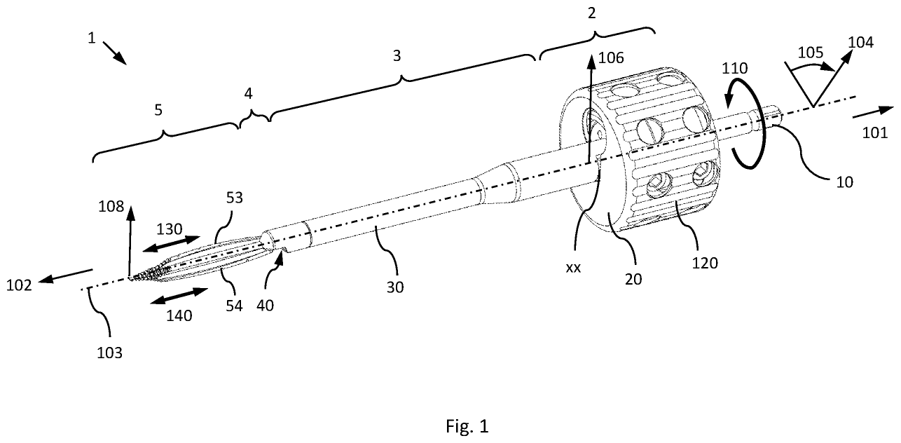

FIG. 1 shows the general structure of the drilling instrument (1) according to the invention. It consists of a gear section (2), a shaft area (3) adjacent thereto, a drill section (5) terminating in the distal direction (102) and a connecting section (4) located therebetween. In the proximal direction (101), there is a rotor (10) which, with the aid of a gear (2), converts a rotational movement (110) into a cyclical translational movement (130, 140) of the shaft elements (30, 40) along the central axis (103). With the aid of a stator (20), the orientation (106) of the drill section (5, 108) and also of the shaft area (3) can be adjusted and retained (120). Thus, the rotor (10) rotates with respect to all instrument components that have the same orientation (106) with the stator (20).

The shaft area (3) preferably extends longitudinally along a central axis (103). This makes it possible to define a main orientation and thus also the central axis (103) on the instrument (1). A longitudinal extension of the shaft area (3) has the advantage of being able to generate openings in bones that are otherwise difficult to reach through the enclosed soft tissue. The shaft area (3) consists of at least two shaft elements (30, 40). The drill section (5) has individually deflectable or movable drilling elements (53, 54) which are in contact with one another but are not fixedly connected with one another. Each shaft element (30, 40) is thereby mechanically connected to a drilling element (30 with 53 and 40 with 54). A movement (e.g. translation) deflects the respective drilling element (53, 54). If two shaft elements (30, 40) are provided, the drilling elements (53, 54) preferably move in counter-rotation for drilling. In this way, openings in the distal direction (102) can be provided without the rotation of the drilling elements (53, 54). The openings are rammed or rasped vertically into the bone.

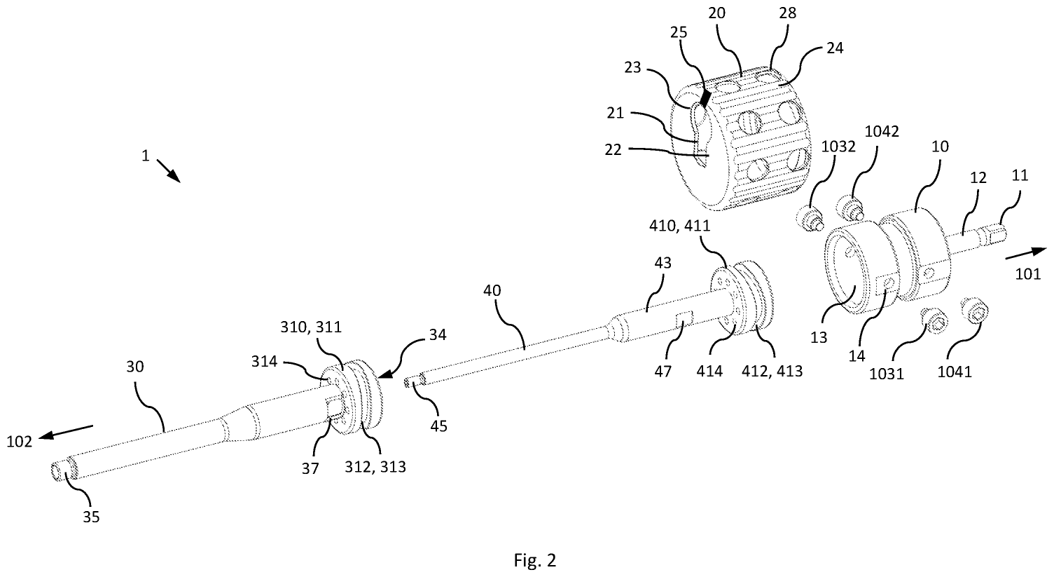

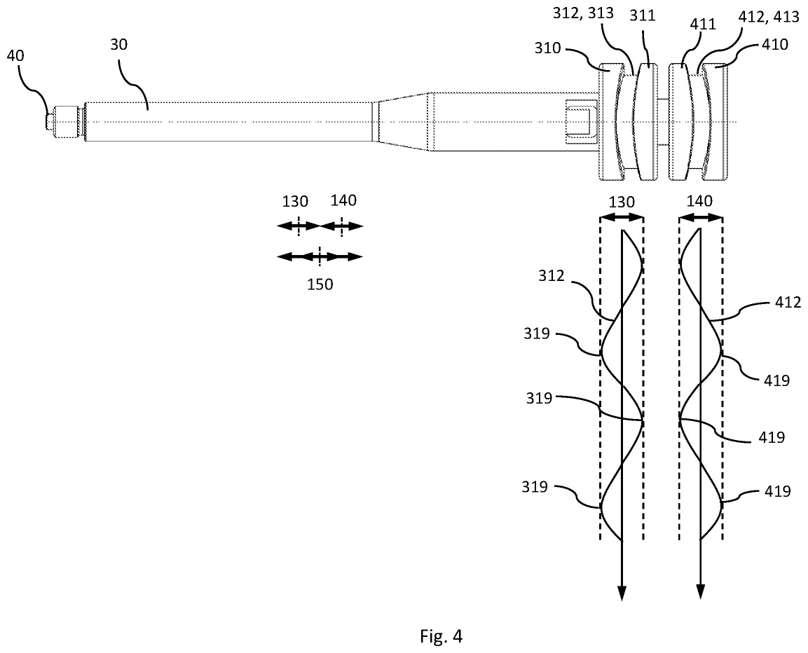

FIG. 2 shows the detailed structure of the drilling instrument (1) according to the invention in an exploded view. In this embodiment, the second shaft element (40) is almost completely inserted into the first shaft element (30). Both shaft elements (30, 40) are designed as a tube, which reduces the manufacturing costs. At the respective distal end of the shaft elements, there is a threaded section (35, 45) which serves as a releasable connection to the drilling elements (53, 54), not depicted here. Both shaft elements (30, 40) each have a cam disc (310, 410) with a lateral surface (311, 411) that is directed outwardly. Further details on the cam discs (310, 410) with their lateral surfaces (311, 411), recesses (313, 413) and their curved lines (312, 412) are explained in more detail in FIG. 4. Both cam discs (310, 410) are rotationally and translationally supported in the rotor (10). This results in a counter-rotating translational movement (130, 140) within the rotor (10), which leads to a cyclical shortening and spacing between the walls of the cam discs that are perpendicular to the central axis (103). This compresses and expands the enclosed spaces. Various openings (314, 414) are preferably provided, which serve in this regard to ventilate these closed-off spaces and to prevent a blocking pumping effect.

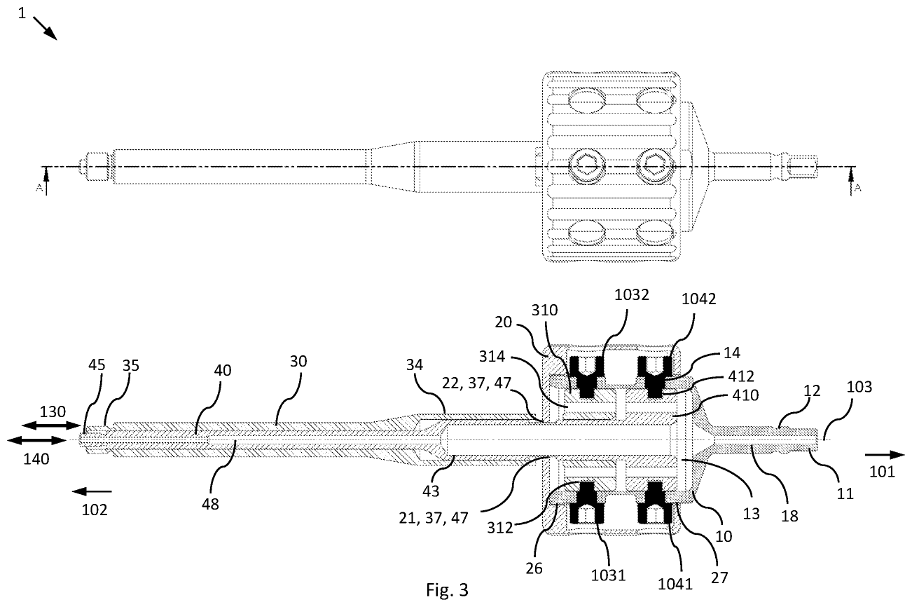

Furthermore, FIG. 2 shows details of the rotor (10). The rotor (10) is mainly round and has a concentric opening (13) that is open in the distal direction (102). In the proximal direction (101), the rotor (10) is preferably closed and merges into a shaft (12). A drive section (11), which contains elements of a quick coupling, is provided on this shaft (12). This drive section (11) is suitable to this end for being able to connect different drive mechanisms that transmit a rotational movement (110) to the rotor (10). Two or more openings (14) are provided on the rotor (10), which are oriented in the radial direction (104) and taper in the direction of the central axis (103). Thus, these openings (14) communicate with the concentric opening (13) of the rotor. Two or more pins (1031, 1032, 1041, 1042) are fixed, joined or supported in these openings (14). In the assembled state, the pins protrude into the interior of the concentric opening (13) of the rotor and are in engagement with the cam discs (310, 410), as depicted in FIG. 3. FIG. 2 further shows the essential details of the stator (20). On the stator, there are surfaces or walls (21, 22) which are suitable to this end for being in direct contact with the surfaces or walls (37, 47) of the shaft elements (30, 40). The contact surfaces are thereby designed in such a way that they tolerate a translational relative movement between the stator (20) and the shaft elements (30, 40), but at the same time the orientation (106) in the circumferential direction (105) between the stator and the drill section (108) is kept constant. In the preferred embodiment of the drilling instrument (1), a keyhole-like opening (23) is provided on the stator (20), through which the shaft elements (30, 40) can be inserted therethrough in the assembled combination coming from the proximal direction.

Furthermore, it is advantageous if an indicator or marking (25) is provided on the stator (20), which indicates the orientation of the drill section (5, 108). For manually holding (120) the stator (20), it is advantageous if there are one or more handles or handle features (24, 28) on the stator (20) that promote a retention. In the embodiment shown here, for the assembly of the instrument, the pins (1031, 1032, 1041, 1042) can be inserted through the openings of the stator (28) and fixed to the rotor (10) with the aid of a tool.

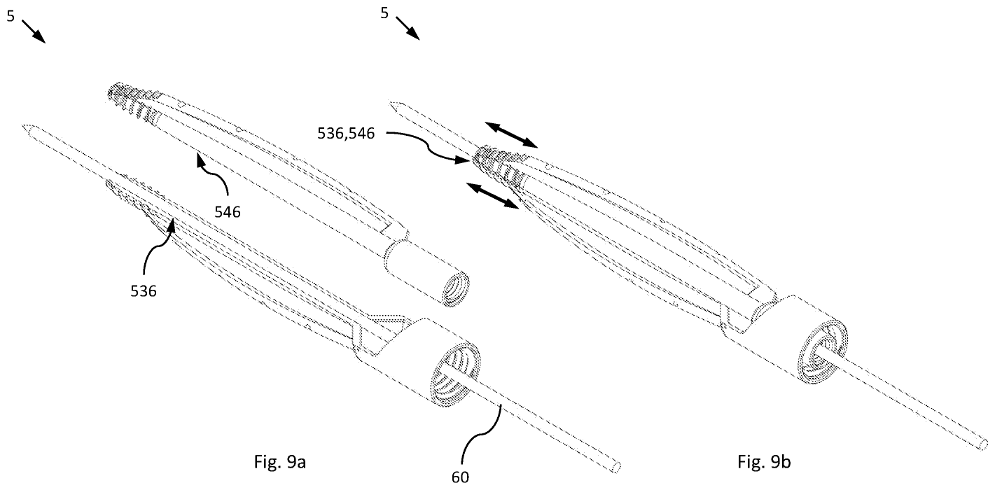

FIG. 3 shows a sectional view of the individual components shown in FIG. 2 and thus allows a view into the assembly and the mode of operation of the drilling instrument (1) according to the invention, the mode of operation and the interaction of the components having already been described at the outset. It can also be seen that the rotor (10) is arranged so as to rotate about the central axis (103). Only through this arrangement it is possible for the entire instrument to be cannulated (18, 48) and for the gear to be able to rotate about an inserted guide wire (60). This is also depicted in FIGS. 9a and b, for example.

In an alternative, but not depicted, embodiment, it would also be conceivable for an additional rotational drilling element to be located within the cannulation (48) and to be joined directly to the rotor (10). The drilling element can thereby protrude distally from the instrument and rotates together with the rotor (10) in relation to the drill section (5). With the internally located rotating drilling element, a centring bore can be generated before the translational drilling area (53, 54) automatically follows.

As depicted in FIG. 4, the cam discs (310, 410) each have a lateral surface (311, 411) that is directed radially outwardly. As a result, the outer diameters of the lateral surfaces 311 and 411 have approximately the same size. Each of the lateral surfaces has a recess (313, 413) directed toward the central axis (103) in the radial direction (104), and this recess extends in the circumferential direction (105) along the lateral surface (311, 411) and forms a self-contained curved line (312, 412). In a developed representation (FIG. 4), this curved line can be represented sinusoidally, it being advantageous for the sine wave (312, 412) to be provided on the lateral surface (311, 411) in an axially symmetrical manner with respect to the longitudinal axis (103). As a result, more than one translational deflection of the shaft elements (30, 40) can be achieved per revolution of the rotor (10). If two or more pins are provided per cam disc in the rotor, at least two or a multiple of two sine waves must be provided per curved line.

For a mutual deflection of the shaft elements (30, 40), it is advantageous if the sinusoidal curved line (312) of the first shaft element (30) is in a phase shift with respect to the sinusoidal curved line (412) of the first shaft element (40). In the embodiment shown, the phase shift optimally corresponds to between 20% and 80%, but preferably approximately 50%, of the oscillation period of the sine wave.

It is advantageous that the curved line (312, 412) is sinusoidal in a developed view and the translation path of the shaft element (130, 140) is defined by the maxima (319, 419) of the sine wave (FIG. 4). With respect to the rotor (10), the translation path of the first and/or second shaft element (130, 140) is between 0.1 mm and 5 mm, preferably 0.2 mm to 4 mm, preferably 0.5 mm to 3 mm. Depending on the phase shift of the cam discs relative to one another, the translation path (150), with respect to the two drilling elements (53, 54) to one another, is the sum of the individual deflections (150=130+140). In the preferred embodiment with two shaft elements (30, 40), the translation path (150) of the first shaft element (30) with respect to the second shaft element (40) is approximately twice the translation path (130, 140) of a shaft element with respect to the rotor (10). This has the advantage that the individual deflections of the shaft elements (130, 140) on the cam discs can be provided to be smaller than actually takes place on the drill section (5, 150) for drilling. This ensures a lower wear function in the gear section (2).

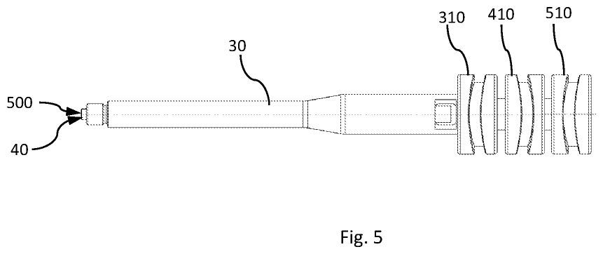

An alternative embodiment is illustrated in FIG. 5. In addition to the preferred variant with two shaft elements (30, 40), more than two shaft elements, as depicted here with three shaft elements (30, 40 and 500), are also conceivable. As a result, any number of shaft elements can be inserted into one another, wherein a cam disc (310, 410 and 510) is provided for each shaft element at the proximal end. The number of the shaft elements also defines the number of drilling elements.

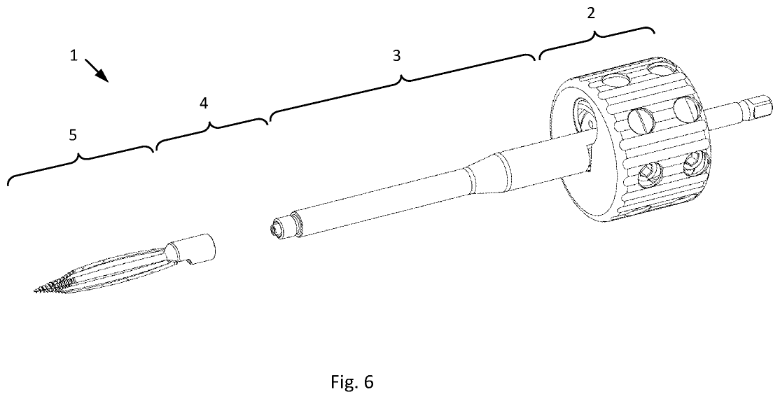

A drill section (5) is provided for drilling with two shaft elements (30, 40) moving in counter-rotation in a translatory cyclical manner to one another, wherein the drill section (5) consists of two drilling elements (53, 54) which are separate from one another but are in direct contact, and a first drilling element (53) is connected to the first shaft element (30) and a second drilling element (54) is connected to the second shaft element (40). It is advantageous for the application if the drilling elements (53, 54) are releasably or interchangeably connected to the shaft elements (30, 40) individually or as a whole (FIG. 6).

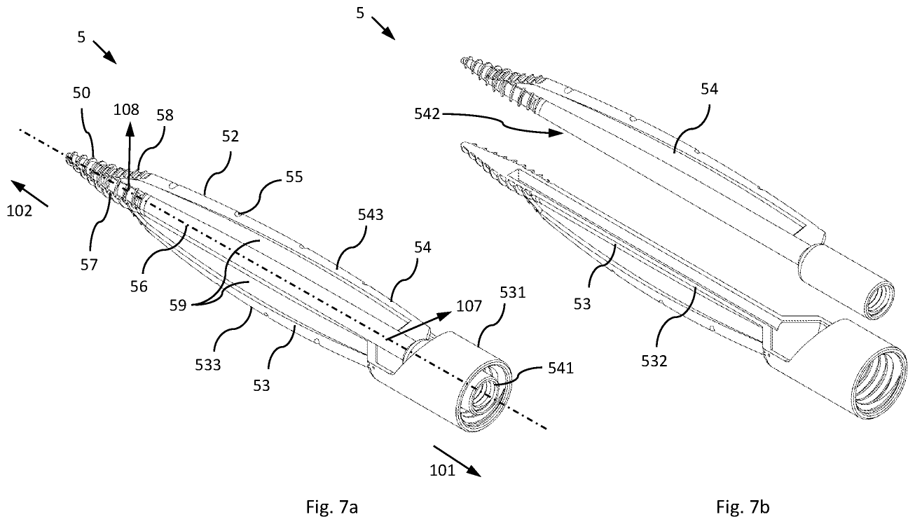

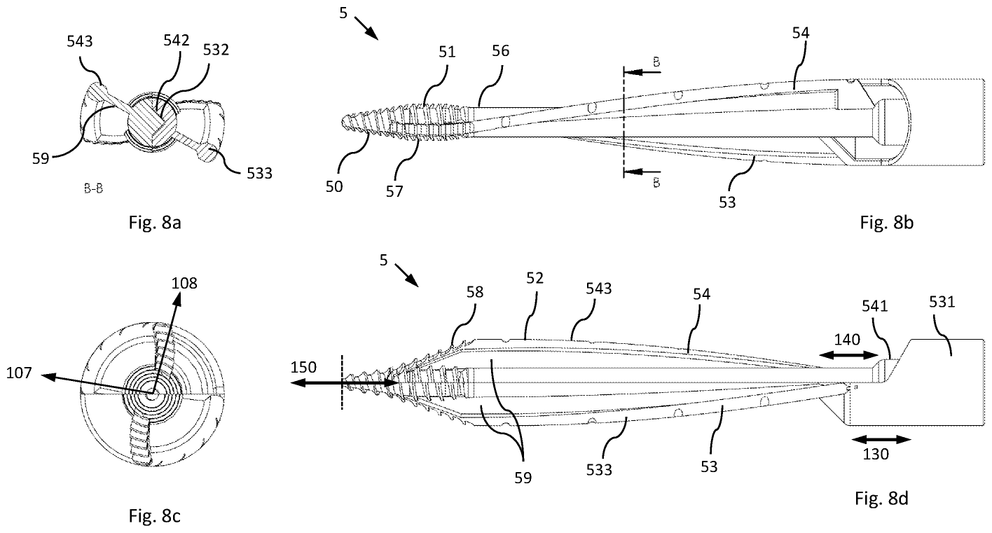

The drilling principle presented here makes it possible to produce openings with any desired cross section, such as round, triangular, square, polygonal or otherwise. For the task set forth above of generating openings for blade-like implants, the drilling instrument (1) presented here also offers the technical possibility of providing openings with the profile of a blade in the bone. Furthermore, it is also possible to generate a helical depth course of the blade profile. In order for a drilling channel for a blade-like implant to be prepared, it is advantageous for the drilling elements (53, 54) to form a blade shape (52) in combination (FIGS. 7a and b) and for at least one of the drilling elements (53, 54) to form at least one blade wing (52). Depicted here are two wings that describe a blade shape overall (52). In the embodiment shown here, the drill section (5) may be divided into two descriptive features; a drill core (56) and the wings (52) adjacent thereto (FIGS. 7a, 7b, 8a-d).

The drill core (56) has a drill bit (50) tapering in the distal direction. A drilling area (51), which adjusts the contour and dimensioning of the core profile to be drilled, adjoins this drill bit (50) in the proximal direction (51). It is also the area with the largest cross section. Adjacent to this area (51) and extending further in the proximal direction, the drill core (56) has a reduced cross section so that this area no longer exerts a drilling effect and the drilling resistance is reduced. Furthermore, material removed in the resulting gap can be transported away in the proximal direction. It is advantageous if at least the drill bit (50) and the drilling area (51) are equipped with rasping teeth (57). Teeth (57) which perform a cutting action in the pulling direction of the drilling elements (53, 54) and can easily be introduced into the material in the pressing direction are particularly suitable for rasping. As depicted (FIGS. 8b and 8d), barb-like rasp teeth are best suited for this purpose. Due to the easier penetration in the pressing direction compared to rasping/machining in the pulling direction, there is also an automatic advance into the material to be drilled. Of course, differently designed rasp or saw teeth with different angles or undercuts are also conceivable.

In addition to the drill core (56) of the drill section (5), wings (52) or a blade shape are provided in the preferred embodiment. The wings (52) preferably have a first proximal orientation (107) and a second orientation in the distal direction (108) (FIGS. 7a, 8c). If a drilling channel for straight blade-like implants is to be provided, both orientations (107, 108) cannot differ. The wings extend mainly parallel along the central axis (103). For curved blade-like implants, however, it is necessary for the orientations (107, 108) to be different from one another and for a helix to be described. The blade-like implant and the drill section should correspond at least partially. This mainly applies to the number of the wings and the defined pitch of the wings. It is also advantageous if the wings (52) merge into the drill core (56) in the distal direction and rasp teeth (58) are formed at least at this transition area. These rasp teeth (58) are in contact with the material to be drilled and have a cutting and abrasive character. A barb-like profile is also suitable here. With the teeth, the profile of the wings to be drilled is shaped into the material.

Pockets (59) can optionally be provided in or on the wings (52) so that the removed material can escape and be discharged from the drilling channel (FIGS. 8a, 8d). The pockets are separated by externally located edges per wing (533 and 543). Preferably, depth indicators (55) are provided on the drilling elements (53, 54), which give the user an indication of the current drilling depth.

When the drilling elements (130, 140) are cyclically deflected, the cross section of the drilling elements changes due to the shift of the drilling elements to one another (150). In the central position, the cross sectional area is maximum, so that both drilling elements (53, 54) form the drilling channel at this position. A deflection position of the drilling elements starting from this central position results in a distal thrust into the material being made possible when the drilling elements are shifted. The proximally extending drilling element transports the rasped material proximally and is supported at the same time on the inner wall of the drilling channel with the rasp teeth (57, 58), in order to allow an abutment for the opposite drilling element extending distally. This arrangement of the rasp teeth in combination with the translational rasping movement reduces the forces necessary for drilling, since the drilling instrument (1) pulls itself into the material to a certain extent. Furthermore, the drill cross section is reduced during a translational deflection movement from the central position, so that the drilling elements in the drilling channel no longer block each other and space is created for the translational movement in the drilling channel.

For the drilling elements (53, 54) to run oriented to one another, they may have a guide structure or profile (532, 542). Different profiles are suitable for this purpose, which prevent both drilling elements (53, 54) from slipping laterally. This function can also be performed by a guide wire (60) as an additional component. As a result, both drilling elements have a partial segment of a cannulation opening (536, 546) (FIGS. 9a and 9b).

It is important for successful use in the clinic that surgical instruments can be disassembled for cleaning, so that all surfaces and cavities of the individual parts can be rinsed and cleaned. In the assembled state, this is difficult for more complex instruments. Therefore, it is important that at least the gear section with the moving parts can be disassembled. For this purpose, it is advantageous if the pins (1031, 1041, 1032, 1042) are releasable from the rotor (10). This can be achieved either by means of a threading.

Alternatively, other snap-on n or releasable joining methods are conceivable. It is also possible for the pins to be provided in groups of two, or for the rotor to have a multi-part structure, for example, and thus for the shaft elements (30, 40) to be freed by the rotor.

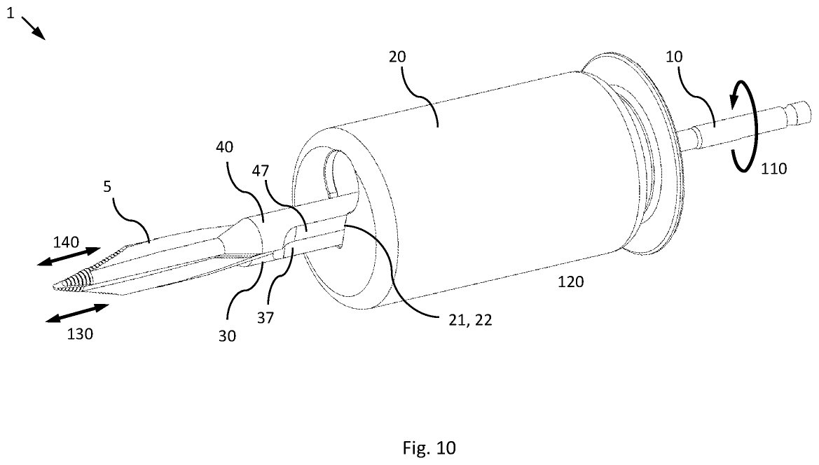

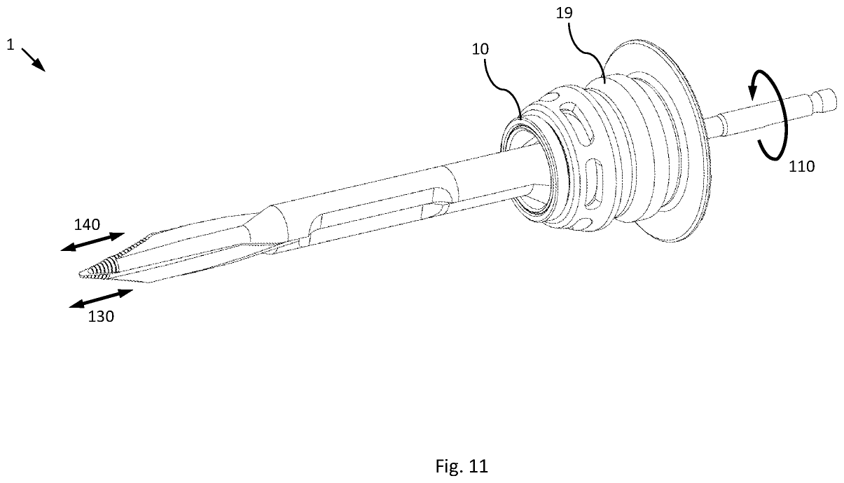

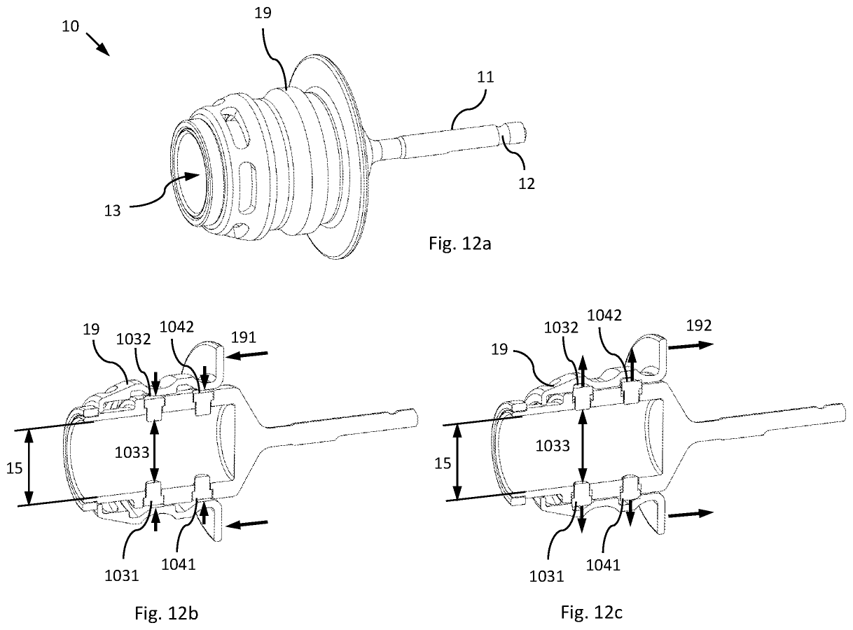

In an alternative embodiment, the rotor (10) has an outer sleeve (19) movable in the longitudinal direction (FIGS. 10-11). As a result, the outer sleeve can assume a first position (191) in which the inner pin distance 1033 is smaller than the inner diameter 130 of the opening of the rotor (13) (FIGS. 12a-c). The outer sleeve (19) can assume a second position (192) in which the inner pin distance 1033 is equal to or greater than the inner diameter 130 of the opening of the rotor (13). That is, once the pins (1031, 1032, 1041, 1042) are released or moved in the radial direction (104), the shaft elements (30, 40) can be removed from the rotor (10).

With this alternative embodiment it is also depicted how the structure is configured in which the first shaft element (30) is supported only partially in the second shaft element (40) (FIGS. 12a and 12b). With the aid of this embodiment, it is possible for both shaft elements (30, 40) themselves to already have surfaces or walls (39, 49) which are in direct contact with one another and prevent both shaft elements from twisting against each other. It is also illustrated that the drilling elements (53, 54) can be configured in one piece with the shaft elements (30, 40), if the manufacturing strategy allows it.

1. A surgical instrument for drilling into bones or bone fragments, comprising a drill section, a gear section and a longitudinal shaft section located therebetween, having a first and a second shaft element, wherein the second shaft element is supported at least partially in the first shaft element, and each shaft element performs a cyclic translational movement along the longitudinal axis for drilling.

2. The surgical instrument according to, claim 1, wherein the first shaft element has at least one opening that is completely enclosed partially along the longitudinal direction but in the circumferential direction.

3. The surgical instrument according to claim 1, wherein the second shaft element has a concentric outer wall at least partially, and the concentric outer wall of the second shaft element is inserted into the opening of the first shaft element.

4. The surgical instrument according to claim 1, wherein the first shaft element is at least partially tubular.

5. The surgical instrument according to claim 1, wherein the second shaft element is designed at least partially tubular or as a full-round rod.

6. The surgical instrument according to claim 1, wherein that both shaft elements each form a cam disc at their proximal end.

7. The surgical instrument according to claim 1, wherein that the first and second shaft elements each form a cam disc, and the cam disc of the second shaft element is arranged in the proximal direction towards the cam disc of the first shaft element.

8. The surgical instrument according to claim 1, wherein the cam discs each have a lateral surface that is directed radially outwardly, and the outer diameters of the lateral surfaces and have approximately the same size.

9. The surgical instrument according to claim 1, wherein the cam discs each have a lateral surface, and the lateral surfaces have a recess directed towards the central axis in the radial direction, and this recess extends in the circumferential direction along the lateral surface and forms a self-contained curved line.

10. The surgical instrument according to claim 9, wherein the curved line is sinusoidal in a developed view and the translation path of the shaft element is defined by the maxima of the sine wave.

11. The surgical instrument according to claim 1, wherein with respect to the rotor, the translation path of the first and/or second shaft element is between 0.1 mm and 5 mm.

12. The surgical instrument according to claim 1, wherein the translation path of the first shaft element with respect to the second shaft element corresponds approximately to twice the translation path of a shaft element with respect to the rotor.

13. The surgical instrument according to claim 9, wherein the curved line is sinusoidal in a developed view, and the sine wave is provided on the lateral surface in an axially symmetrical manner with respect to the longitudinal axis.

14. The surgical instrument according to claim 1, wherein the sinusoidal curved line of the first shaft element is in a phase shift with respect to the sinusoidal curved line of the first shaft element.

15. The surgical instrument according to claim 14, wherein the phase shift corresponds to between 20% and 80% of the oscillation period of the sine wave.

16. The surgical instrument according to claim 1, wherein the shaft elements are rotationally and translationally supported at their proximal ends with the cam discs within a rotor.

17. The surgical instrument according to claim 1, wherein the rotor has at least two pins and at least one pin is in engagement in the recess of the first cam disc and at least one further pin is in engagement in the recess of the second cam disc.

18. The surgical instrument according to claim 17, wherein the rotor has two circumferentially opposite pins that are in engagement in the recess of the first cam disc, and the rotor has two further circumferentially opposite pins that are in engagement in the recess of the second cam disc.

19. The surgical instrument according to claim 1, wherein the pins as well as the pins are placed at an angle to one another in the circumferential direction.

20. The surgical instrument according to claim 1, wherein the pins are releasable from the rotor.

21. The surgical instrument according to claim 1, wherein the rotor has an outer sleeve movable in the longitudinal direction, and this outer sleeve can assume a first position in which the inner pin distance is smaller than the inner diameter of the opening of the rotor, and the outer sleeve can assume a second position in which the inner pin distance is equal to or greater than the inner diameter of the opening of the rotor.

22. The surgical instrument according to claim 1, wherein once the pins are released or displaced in the radial direction, the shaft elements can be removed from the rotor.

23. The surgical instrument according to claim 1, wherein the rotor has a proximally extending shaft area which provides a coupling connection which is suitable to this end for transmitting a torque to the rotor.

24. The surgical instrument according to claim 1, wherein the shaft elements are in contact to one another directly or indirectly along the longitudinal axis on at least one section, and a twist of the shaft elements against each other is thus excluded.

25. The surgical instrument according to claim 1, further comprising a stator configured to serve as a reference for orienting the drill section.

26. The surgical instrument according to claim 25, wherein the stator has an indicator which indicates the orientation of the drill bit.

27. The surgical instrument according to claim 25, wherein the stator has at least one handle.

28. The surgical instrument according to claim 25, wherein a surface or projection which is in contact with the stator is provided on at least one of the shaft elements such that the shaft element with the stator are always at the same angle to one another in the circumferential direction.

29. The surgical instrument according to claim 1, wherein the entire drilling instrument, including the drill section, is continuously cannulated, and a guide wire can be accommodated therein.

30. The surgical instrument according to claim 1, wherein the drill section consists of two drilling elements which are separate from one another but are in direct contact, and a first drilling element is connected to the first shaft element and a second drilling element is connected to the second shaft element.

31. The surgical instrument according to claim 1, wherein the drilling elements are releasably connected to the shaft elements.

32. The surgical instrument according to claim 1, wherein the drilling elements in combination generate openings in the bone that have a non-round cross section.

33. The surgical instrument according to claim 1, wherein the drilling elements in combination generate openings in the bone that deviate from the central axis along the depth.

34. The surgical instrument according to claim 1, wherein the drilling elements in combination form a blade shape.

35. The surgical instrument according to claim 1, wherein the drilling elements in combination generate openings in the bone, and the opening has a first cross section and a second cross section at two different depths, and the orientations of the both cross sections are different.
